(19) United States
(12) Patent Application Publication
(10) Pub. No.: US 2024/0392235 A1
(43) Pub. Date: Nov. 28, 2024

(54) PSEUDOMONAS AND USE THEREOF

(71) Applicants: CHINA PETROLEUM & CHEMICAL CORPORATION, Beijing (CN); INSTITUTE OF PETROLEUM ENGINEERING, SHENGLI OILFIELD COMPANY, CHINA PETROLEUM & CHEMICAL CORPORATION, Dongying, Shandong (CN)

(72) Inventors: Weidong WANG, Dongying, Shandong (CN); Jing HU, Dongying, Shandong (CN); Yanbin CAO, Dongying, Shandong (CN); Qin QIAN, Dongying, Shandong (CN); Lei ZHANG, Dongying, Shandong (CN); Liaoyuan GUO, Dongying, Shandong (CN); Gongze CAO, Dongying, Shandong (CN); Gangzheng SUN, Dongying, Shandong (CN); Junzhang LIN, Dongying, Shandong (CN); Yongting SONG, Dongying, Shandong (CN); Shenghui YUE, Dongying, Shandong (CN); Changzhong YUAN, Dongying, Shandong (CN); Xiaoling WU, Dongying, Shandong (CN); Mingshan DING, Dongying, Shandong (CN); Guangjun GAO, Dongying, Shandong (CN); Jing WANG, Dongying, Shandong (CN); Tao LIU, Dongying, Shandong (CN); Yun FENG, Dongying, Shandong (CN); Caifeng LI, Dongying, Shandong (CN); Xin SONG, Dongying, Shandong (CN); Zihui CHEN, Dongying, Shandong (CN)

(21) Appl. No.: 18/692,975

(22) PCT Filed: Sep. 16, 2022
(86) PCT No.: PCT/CN2022/119404

§ 371 Date: Mar. 18, 2024

(30) Foreign Application Priority Data
Sep. 18, 2021 (CN) 202111101957.1

(51) Int. Cl. C12N 1/20; C09K 8/582; C12R 1/38

(52) U.S. Cl. CPC C12N 1/205; C09K 8/582; C12R 2001/38

(57) ABSTRACT

Pseudomonas and a use thereof are provided. The preservation number of the Pseudomonas is CGMCC No. 22583. The growth temperature of the Pseudomonas is 37-45° C. Under a medium-high temperature oil reservoir condition, the Pseudomonas can be denatured from a non-viscosity fluid to a multifunctional oil-extraction bacterial solution having both surface activity and viscoelasticity, and also has the functions of expanding the swept volume and improving the oil washing efficiency. The recovery ratio is increased by more than 20% in a physical simulation experiment.

### CROSS-REFERENCE TO RELATED APPLICATIONS

The application claims the benefit of Chinese patent application No. “202111101957.1”, filed on Sep. 18, 2021, the content of which is specifically and entirely incorporated herein by reference.

### TECHNICAL FIELD

The invention belongs to the field of energy biotechnology and environmental biotechnology, and particularly relates to a Pseudomonas and a use thereof.

### BACKGROUND ART

After most oil fields have been developed by applying the conventional oil production technology, a part of crude oil still retains in the oil reservoir and cannot be extracted, but the oil field has entered a high water content development stage. How to improve the recovery ratio of the oil field in the high water content development stage is critical for the sustainable and stable production of the oil field.

The microbial oil recovery technology utilizes the microbes which have an oil displacement function to be injected into an oil reservoir or activated in the oil reservoir and improves the recovery ratio of the crude oil through the growth and metabolic activities of the microbes, and the interaction of the metabolites generated by the microbes with the crude oil and the reservoir. A large number of laboratory investigations and field tests have been implemented around the world, although the technology exhibits the desirable application prospects, it has not been popularized and applied on a large scale on the field. In view of the complexity of the oil reservoir development, the crude oil recovery ratio can only be effectively improved by simultaneously improving the oil washing efficiency and expanding the swept volume, while the existing microbial oil recovery technology mainly comprises the following kinds: activating the endogenous microorganism or injecting the exogenous microorganism, however, the complexity of endogenous microorganism causes that it is difficult to accurately activate and regulate the target bacterial colony, and the exogenous microorganism has the limitation of single function and cannot effectively solve various contradictions in an oil reservoir, so that the development of multifunctional microorganism strains capable of simultaneously expanding the swept volume (with an apparent viscosity more than 50 mPa·s) and improving the oil washing efficiency is critical for enhancing the effects of implementing the microbial oil recovery technology. Ming ZHAO, et al. (“Screening, Evaluation and Field Application of a Pseudomonas Strain for Oil Production”, Shandong chemical industry, Vol. 50, 2021, the publication date is Aug. 8, 2021) obtains a Pseudomonas strain NGD, but the fermentation broth only has the function of washing oil, and the highest oil washing efficiency is 77%.

### SUMMARY OF THE INVENTION

The present invention aims to overcome the aforementioned defects in the prior art and provides a pseudomonad capable of expanding the sweep volume and improving the oil washing efficiency and the use thereof.

The first aspect of the invention provides a Pseudomonas (Pseudomonas sp.) strain, of which the preservation number is CGMCC No. 22583.

The second aspect of the present invention provides a use of the aforesaid Pseudomonas and/or metabolite thereof in oil recovery.

The third aspect of the present invention provides a method for recovering oil, the method comprises the following steps: injecting an oil-extraction bacterial solution into an oil reservoir to produce oil, wherein the oil-extraction bacterial solution contains at least one selected from the group consisting of a thallus of the aforesaid Pseudomonas, an extracellular metabolite of the Pseudomonas, and an intracellular metabolite of the Pseudomonas.

The invention at least produces favorable technical effects as follows:
(1) The growth temperature of the Pseudomonas Pse-1 provided by the invention is within a range of 37-45° C., and its fermentation broth can be denatured from a non-viscosity fluid to a multifunctional oil-extraction bacterial solution having both surface activity and viscoelasticity under a medium-high temperature (60-95° C.) oil reservoir condition, and also has the functions of expanding the swept volume and improving the oil washing efficiency; after being denatured under the oil reservoir temperature of 60-95° C., the multifunctional oil-extraction bacterial solution has an apparent viscosity more than or equal to 50 mPa·s, a surface tension less than or equal to 30 mN/m and an oil washing efficiency more than or equal to 90%; in addition, the recovery ratio is increased by more than 20% in the physical simulation experiment.
(2) When the fermentation broth of Pseudomonas Pse-1 is applied to the microbial huff and puff, the average daily oil production of a single well is increased by more than 50%, the oil production per ton agent is increased by more than 20 t, the validity period is more than 250 d, and the output-input ratio is larger than 5;
(3) When the fermentation broth of the Pseudomonas Pse-1 is applied to the microbial oil displacement, the recovery ratio of an oil well group is finally improved by more than 8%, the oil production per ton agent is increased by more than 15 t, the validity period is more than 350 d, and the output-input ratio is larger than 3.

### BIOLOGICAL PRESERVATION

The classification and nomenclature of the strains of the invention is Pseudomonas (Pseudomonas sp.), which has been preserved in the China General Microbiological Culture Collection Center (CGMCC) (Address: Institute of Microbiology, Chinese Academy of Sciences, No. 3, Courtyard 1, Beichen West Road, Chaoyang District, Beijing 100101, China), and of which the preservation number is CGMCC No. 22583.

### BRIEF DESCRIPTION OF THE DRAWINGS

The accompanying drawings are provided to facilitate further comprehension of the invention, and constitute a part of the description, the accompanying drawings and the DESCRIPTION OF THE PREFERRED EMBODIMENT described below serve to explain the invention and do not impose restriction thereto. In the accompanying drawings:

FIG. 1 is a phylogenetic tree diagram of a Pseudomonas Pse-1 strain disclosed in the invention;

FIG. 2 illustrates a colony morphology graph of a Pseudomonas Pse-1 strain disclosed in the invention;

FIG. 3 shows a morphological diagram of a Pseudomonas Pse-1 strain disclosed in the invention after staining the thallus;

FIG. 4 is a morphological diagram of a network-like polysaccharide structure in the bacterial solution of a Pseudomonas Pse-1 strain disclosed in the invention after denaturing at 60° C.;

FIG. 5 illustrates a schematic diagram of the viscosity change of a Pseudomonas Pse-1 strain fermentation broth (with a dilution of 50%) disclosed in the invention over different times at 60° C.

### DESCRIPTION OF THE PREFERRED EMBODIMENT

The terminals and any value of the ranges disclosed herein are not limited to the precise ranges or values, such ranges or values shall be comprehended as comprising the values adjacent to the ranges or values. As for numerical ranges, the endpoint values of the various ranges, the endpoint values and the individual point value of the various ranges, and the individual point values may be combined with one another to produce one or more new numerical ranges, which should be deemed have been specifically disclosed herein.

Unless otherwise specified in the present invention, the term “fermentation product” refers to the entire product obtained without performing any separation procedure after culturing the pseudomonads, and includes the pseudomonad thallus and the metabolic products of the pseudomonad.

The first aspect of the invention provides a Pseudomonas (Pseudomonas sp.) strain, characterized in that its preservation number is CGMCC No. 22583.

The 16S rDNA sequence of the Pseudomonas of the invention is as shown by SEQ ID NO: 1, thus the invention also relates to a Pseudomonas strain with a preservation number of CGMCC No. 22583, or the 16S rDNA sequence thereof shown by SEQ ID NO: 1.

The second aspect of the invention provides a use of the aforementioned Pseudomonas and/or metabolite thereof in oil recovery.

The third aspect of the invention provides a method for recovering oil, characterized in that the method comprises the following steps: injecting an oil-extraction bacterial solution (or an oil recovery agent) into an oil reservoir to produce oil, wherein the oil-extraction bacterial solution (or an oil recovery agent) contains a thallus and/or metabolite (including the intracellular metabolite and/or extracellular metabolite) of the aforesaid Pseudomonas.

The Pseudomonas of the present invention is capable of producing metabolites (extracellular metabolite and/or intracellular metabolite, possibly including biosurfactants and/or temperature-sensitive viscoelastic biopolysaccharides) that are beneficial for oil recovery. Different forms of the oil recovery bacterium solution containing said Pseudomonas metabolite may be used, for example, the oil recovery bacterium solution may contain at least one of the thallus, extracellular metabolite of said Pseudomonas and thallus lysate (or intracellular metabolite released from the thallus lysis).

In the invention, the thallus of the Pseudomonas can be live thallus or dead thallus, as long as the metabolite generated in the thallus growth (fermentation) process is at least partially retained in the oil-extraction bacteria solution, which can contact with an oil reservoir to displace oil when in use. The metabolites which are generated by the Pseudomonas and are beneficial to oil extraction comprise extracellular metabolites and intracellular metabolites, such that the oil-extraction bacteria solution containing thallus can be directly injected into an oil reservoir, and the thallus are lysed in the oil reservoir to secrete the intracellular metabolites and the extracellular metabolites to jointly promote oil extraction. The oil reservoir temperature of the medium-high temperature oil reservoir is relatively high, and the injected thalli will be lysed under the action of the surfactant generated from the extracellular metabolism, thus the oil extraction mode of directly injecting the oil-extraction bacterial solution containing the thalli is particularly suitable for the medium-high temperature oil reservoir. Nevertheless, it is also feasible to lyse the thallus in advance (e.g., perform a heating and lysis process) and inject the resulting lysate into the oil reservoir for oil extraction, it is understandable that the mode of lysing the thallus in advance is particularly suitable for the low-temperature oil reservoirs (lower than 60° C.). Therefore, the method of the invention can further comprise the steps of preparing an oil-extraction bacterial solution: (i) inoculating the Pseudomonas into a nutrient medium for fermentation; and optionally, (ii) subjecting the resulting fermentation product to a thallus lysis operation, or separating the thallus from the fermentation product and subjecting the obtained thallus to lysis. The fermentation product obtained according to step (i) contains thalli and can be directly used for medium-high temperature oil reservoirs. The lysate obtained according to step (ii) comprises metabolites of Pseudomonas and is suitable for the low-temperature oil reservoirs.

In step (i), the nutrient medium comprises a carbon source, a nitrogen source and an inorganic salt. The initial pH of the nutrient medium is within a range of 7.5-8.

Preferably, the nutrient medium contains the carbon source in an amount of 20-50 g/L, more preferably 23-47 g/L. The carbon source may be a commonly used carbonaceous material for culturing Pseudomonas, preferably xylose and/or glycerol, more preferably xylose and glycerol. Further preferably, the weight ratio of glycerol to xylose is within a range of 1-5 (e.g. 1, 1.2, 1.5, 1.8, 2, 2.5, 3, 3.5, 4, 4.5, 5, or any value within a range formed by two numerical values thereof). Most preferably, the content of xylose in the nutrient medium is 8-12 g/L. Most preferably, the content of glycerol in the nutrient medium is 15-35 g/L.

Preferably, the nutrient medium contains the nitrogen source in an amount of 0.5-2 g/L. The nitrogen source may be a nitrogen-containing substance commonly used for culturing Pseudomonas, preferably yeast extract powder.

Preferably, the nutrient medium contains the inorganic salt in an amount of 3-12 g/L, more preferably 3.3-10.8 g/L. The inorganic salt may be an inorganic salt commonly used for culturing Pseudomonas, including at least one selected from the group consisting of sodium salt, potassium salt, calcium salt and magnesium salt, for example, the inorganic salt may be at least one selected from the group consisting of sodium nitrate, potassium nitrate, dipotassium phosphate, disodium phosphate, sodium chloride, potassium chloride, calcium chloride, magnesium sulfate and magnesium chloride, preferably at least one selected from the group consisting of sodium nitrate, dipotassium phosphate, sodium chloride, calcium chloride and magnesium sulfate.

According to a particularly preferred embodiment of the invention, the nutrient medium comprises 8-12 g/L of xylose, 15-35 g/L of glycerol, 1-4 g/L of sodium nitrate, 1-3 g/L of dipotassium phosphate, 1-3 g/L of sodium chloride, 0.05-0.1 g/L of calcium chloride, 0.3-0.7 g/L of magnesium sulfate, 0.5-2 g/L of yeast extract powder, and an initial pH of the nutrient medium is within a range of 7.5-8. More Pseudomonas and/or their metabolites can be obtained by adopting the culture medium of the preferred embodiment, which is conducive to further reducing the energy or material consumption for oil recovery.

In step (i), the fermentation can be carried out under the conventional condition of culturing the Pseudomonas, preferably, the fermentation condition facilitates that the content of the Pseudomonas in the fermentation broth is more than or equal to 109 CFU/mL, or the fermentation condition satisfies that the fermentation broth after being heated at 60° C. and above for 8 h has a surface tension less than or equal to 30 mN/m and an apparent viscosity more than or equal to 50 mPa·s. More preferably, the fermentation conditions comprise: the inoculation amount within a range of 10⁶-10⁸ CFU/mL. The fermentation temperature is preferably within a range of 37-45° C. The fermentation time is preferably within a range of 48-72 h. The aeration rate of the fermentation is preferably within a range of 1-2 L/min.

In the present invention, a step of seed culture may be generally performed before the fermentation in step (i) to amplify the Pseudomonas. The seed medium used for seed culture may comprise: 2-4 g/L of glucose, 2-4 g/L of peptone, and 2-4 g/L of yeast extract powder. The condition of seed culture can be shake culture at the temperature of 37-45° C. and a rotational speed of 150-200 r/min for 20-30 h.

In step (ii), the whole fermentation product can be directly subjected to lysing operation, so that thalli in the fermentation product are lysed to obtain a thalli lysate; alternatively, the thalli may be separated from the fermentation product and then lysed to obtain the thallus lysate. Lysis mainly serves to disrupt the thalli and release intracellular metabolites, it can be performed by using the conventional methods in the art, for example, by using the high-temperature lysis process (e.g., treating at 60-95° C. for 8-10 h), repeated freeze-thaw method, ultrasonic treatment method, or carrying out lysing by means of a lysate.

In the invention, the surface tension of the oil-extraction bacteria solution at 25° C. is less than or equal to 30 mN/m, and preferably within a range of 24.5-28.5 mN/m. The apparent viscosity of the oil-extraction bacteria solution at 60° C. is more than or equal to 50 mPa·s, and preferably within a range of 65-150 mPa·s. Wherein the surface tension can be measured by referring to a flat plate method specified in the China National Standard GB/T22237-2008. The apparent viscosity can be measured by using a Brook field rotational viscometer (rotor model: rotor No. 3; rotor speed: 60 r/min) (referring to the China National Standard GB 1886.41-2015). The fermentation product obtained in step (i) and the thallus lysate obtained in step (ii) can be diluted with a diluent and then be used for oil recovery, the diluent is preferably used in an amount such that the surface tension and/or apparent viscosity of the oil-extraction bacteria solution satisfy the above requirements, and in order to satisfy the above requirements, the dilution degree (=diluent volume/(fermentation product volume+diluent volume)×100%) of the fermentation product obtained in step (i) and/or the thallus lysate obtained in step (ii) is generally not higher than 50%. The diluent is preferably tap water or formation water, more preferably formation water, in particular the formation water with a degree of mineralization of 1,000-100,000 mg/L.

In the invention, the mode of injecting an oil-extraction bacterial solution into an oil reservoir to produce oil may be an oil well huff and puff. The Pseudomonas of the present invention is advantageous to shorten the closed well cultivation time, therefore, when the oil well huff and puff is adopted, the closed well cultivation time can be 10⁻³⁰ d, preferably 15-30 d. The injection speed of the oil-extraction bacteria solution can be 10-20 m³/h, and the oil well is opened for production after the closed-well cultivation.

The invention does not impose a particular requirement on the total injection volume of the oil-extraction bacterial solution in the oil well huff and puff, preferably the total injection volume of the oil-extraction bacterial solution satisfies the formula V=πR²Hϕβ, wherein:
V denotes the total injection volume of the oil-extraction bacterial solution, the unit is m³;
R denotes the treatment radius, the unit is m;
H denotes the effective thickness, the unit is m;
ϕ denotes the porosity;
B denotes the dosage coefficient, the value is within a range of 1-1.5.

In the invention, the mode of injecting an oil-extraction bacterial solution into an oil reservoir to produce oil may be a water injection well displacement, where an oil-extraction bacterial solution is injected from a water injection well in a slug manner, as a consequence, the mode of injecting an oil-extraction bacterial solution into an oil reservoir by means of a well displacement may comprise the following steps:
(1) Injecting an oil-extraction bacterial solution and water into an oil reservoir in sequence;
(2) Stopping the injecting operation;
(3) Injecting water into the oil reservoir;
(4) Circularly performing the steps (1)-(3) at least twice.

Preferably, the used amount of the oil-extraction bacterial solution in step (1) is 14-50% of the total injection volume of the oil-extraction bacterial solution. The total injection volume of the oil-extraction bacterial solution is within a range of 0.05-0.1 PV (pore volume). Wherein PV denotes the pore volume of the reservoir stratum where the oil reservoir is located, the pore volume is the total volume of the effective pores in the reservoir stratum, and the pore volume is obtained by calculating the product of multiplying the reservoir stratum volume with the porosity. Wherein the porosity can be measured according to the method stipulated in the Petroleum Industrial Standard SY/T6298-1997 of China after the core extraction from the oil reservoir. For the oil well group with a well spacing of 200-300 m, the injection speed of the oil-extraction bacteria solution in step (1) may be within a range of 5-20 m³/h. In general, both the oil-extraction bacteria solution and water are injected through the water injection well.

Preferably, the water injection volume in steps (1) and (3) is 14-50% of the total water injection volume. The ratio of the water injection volume in step (1) to the water injection volume in step (3) is preferably 1:0.05-0.7. The total water injection volume is preferably 0.1-0.3 PV (pore volume). For the oil well group with a well spacing of 200-300 m, the injection speed of water in both step (1) and step (3) can be 10-50 m³/h. Both the oil-extraction bacteria solution and water are injected through the water injection well.

Preferably, the time of stopping the injection operation in step (2) is 1-2 d.

Preferably, the circular times performed in step (4) is 2-7.

In the invention, the temperature of an oil reservoir can fluctuate within a wide range. When the oil reservoir is a medium-high temperature oil reservoir, the temperature of the oil reservoir is generally within a range of 60-95° C. When the oil reservoir is a low-temperature oil reservoir, the temperature of the oil reservoir is generally lower than 60° C. (such as 30-59° C.). The viscosity of the dehydrated and degassed crude oil in the oil reservoir at the temperature condition of 50° C. is less than or equal to 10,000 mPa·s (e.g., 1,500-4,000 mPa·s). The viscosity of crude oil can be measured at 50° C. by using a rotational viscometer with reference to the method stipulated in the Petroleum Industrial Standard SY/T0520-2008 of China.

The method of the invention is capable of expanding the swept volume and improving the oil washing efficiency simultaneously, therefore, the invention also relates to a method of expanding the swept volume and improving the oil washing efficiency simultaneously, characterized in that the method comprises the following steps: injecting the oil-extraction bacterial solution into an oil reservoir for oil recovery, wherein the oil-extraction bacterial solution is as described above, the content will not be repeatedly described herein.

The present invention will be described in detail below with reference to examples.

The nutrient medium (nutrient solution) used in Examples 1, 2 and 4-7 comprised the following ingredients: 10 g/L of xylose, 30 g/L of glycerol, 2 g/L of sodium nitrate, 2 g/L of dipotassium phosphate, 2 g/L of sodium chloride, 0.1 g/L of calcium chloride, 0.5 g/L of magnesium sulfate, 1 g/L of yeast extract powder, a pH of 7.8, and the balance was water.

The seed liquid culture medium (nutrient solution) comprised the following ingredients: 3 g/L of glucose, 3 g/L of peptone and 3 g/L of yeast extract powder; the seed liquid was obtained by the following steps: 1 mL of strain cryopreservation tube was inoculated into 100 mL of seed liquid culture medium, the shaking culture was performed at 37° C. under aerobic condition and a rotational speed of about 170 r/min for 24 h to obtain the seed liquid, wherein the content of Pseudomonas Pse-1 in the seed liquid was 10⁸ CFU/mL.

The method for obtaining the fermentation broth comprised the following steps: the Pseudomonas Pse-1 seed solution was inoculated into a nutrient medium according to 2 vol. % of the inoculation amount, cultured for 60 h at a temperature of 37° C., a stirring rate of 200 rpm and an aeration amount of 1.5 L/min to obtain the fermentation broth, wherein the content of the Pseudomonas Pse-1 in the fermentation broth was 10⁹ CFU/mL.

The daily oil yields for the oil well and block before and after the oil recovery were calculated by using the conventional metering separator, which was also generally called the glass tube oil measuring method, the method was based on the principle of connecting pipe balance, the liquid volume was measured by using the method of constant volume, in combination with the water content of the produced liquid measured by sampling to calculate the daily oil yield of the oil well or block.

### Example 1

### Acquisition and Identification of Pseudomonas Pse-1 of the Invention

### I. Acquisition of Pseudomonas Pse-1

A Pseudomonas strain, called Pse-1 for short, was separated from the produced fluid of an oil well of the Shengli Oil Field in China.

### II. Identification of Pseudomonas Pse-1

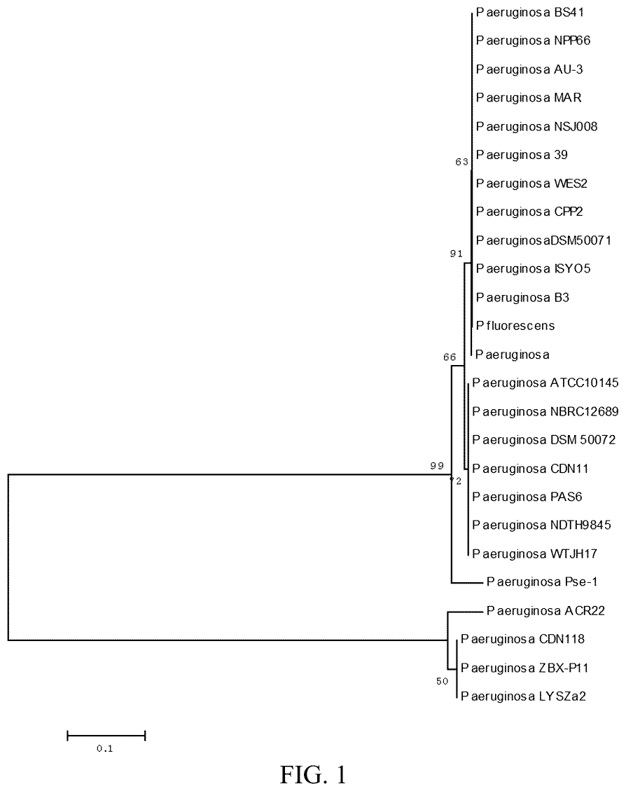

16S rDNA amplification and sequencing were carried out on Pse-1, and the strain identification was performed, the steps were as follows: denaturing, PCR amplification, PCR product purification and target fragment sequencing. The sequence of 16S rDNA was as shown by SEQ ID NO: 1. The sequence comparison was implemented at the NCBI according to the sequencing result, wherein the similarity was 96%, a phylogenetic tree was constructed (FIG. 1), the Pseudomonas Pse-1 of the invention was identified as a new strain belonging to the genus Pseudomonas, its classification and nomenclature were as follows: Pseudomonas (Pseudomonas sp.).

SQE ID NO: 1:
AGCCTACACATGCAAGTCGAGCGGCAGCACGGGTACTTGTACCTGGTGG
CGAGCGGCGGACGGGTGAGTAATGCCTAGGAATCTGCCTGGTAGTGGGG
GATAACGCTCGGAAACGGACGCTAATACCGCATACGTCCTACGGGAGAA
AGCAGGGGACCTTCGGGCCTTGCGCTATCAGATGAGCCTAGGTCGGATT
AGCTAGTTGGTGAGGTAATGGCTCACCAAGGCGACGATCCGTAATTGGT
CTGAGAGGATGATCAGTCACACTGCAACTGAGACACGGTCCAGACTCCT
ACGGGAGGCACGAGTGGGGAATATTGGACAATGGGCGAAAGCCTGATCC
AGCCATGCCGCGTGTGTGAAGAAGGTGTACGGATTGTTAAGCACAATAA
GTTGGGAGGAAGGGCATTTACCTAATACGTAAGTGTTTTGACGTTACCG
ACAGAAAAACCACCAACTACCTCTGTGCCAGCAGCCGCGGTAATACAGA
GGGTGCAAGCGTTAATCGCAATAACTGGGCGTAAAGCGCGCGTAGGTGG
TATGTTAAGTTGGATGTGAAATCCCCGGGCTCAACCTGGGAACTGCATC
CAAAACTCCCAAGCTAGACTATGGTAGAGGGTGGTGGAATTTCCTGTGT
AGCGGTGATATCCGTAGATATCGGAAGGAACACCAGTGGTGAAGGCGAC
CACCTGGACTGATACTGACACTGAGGTGCGAAAGCGTGCGGAGCATACA
GGATTAGAAACCCTGGTAGTCCACGCCGTAAACGATGTCAACTAGCCGT
TGGGTGCCTTGAGCTCTTCGTGGCGCAGGTATCGCATTAAGTTGACCGC
CTGGGGAGTACGGCCGCAAGGTTATAACTCATATGATTTGACGGGGGGG
CGCACAAGCGGTGGAGCATGTGGTTTAATTCGAAGCAACGCGAAGAACC
TTACCAGGCCTTGACATCCAATAAACTTTCCAGAGATGGATTGGTGCCT
TCGGGAACATTGAGACAGGTGGTGCATGCCTGTCGACAGCTCGTGTCGT
GAGATGTTGGGTTAAGTCCCGTAACGAGCGCAACCCTTGTCCTTAGTTA
CCAGCACATTTTGGTCCCCACTCTAAGGAGACTGCCGGTGACAAACCGG
AGGAAGGTGGGGATGACCTCAAGTCATCATGGCCCTTACGGCCTGGGCT
ACACACGTGCTACAATGGTCGGTACAGAGGGTTGCCAAGCCGCGAGGTA
GAGCTAATCCCATAAAACCGATCGTAGTCCGGATCGCAGTCTGCAACTC
GACTGCGTGAAGTCGGAATCGCTAGTAATCGCGTTTCAGAATGTCGCGG
TGAATACGTTCCCGGGCCTTGTACACACCGCCCGTCACACCATCCGAGT
GGGTTGCACCAGAAGTAGCTAGTCTAACCTTCGGGGGGACGGTACCACG
GT

The following aspects were observed with reference to the experimental method of Bergey's Manual of Systematic Bacteriology: thallus size and morphology, presence or absence of flagella and spores, growth temperature, aerobic condition, and bacterial colony morphology.

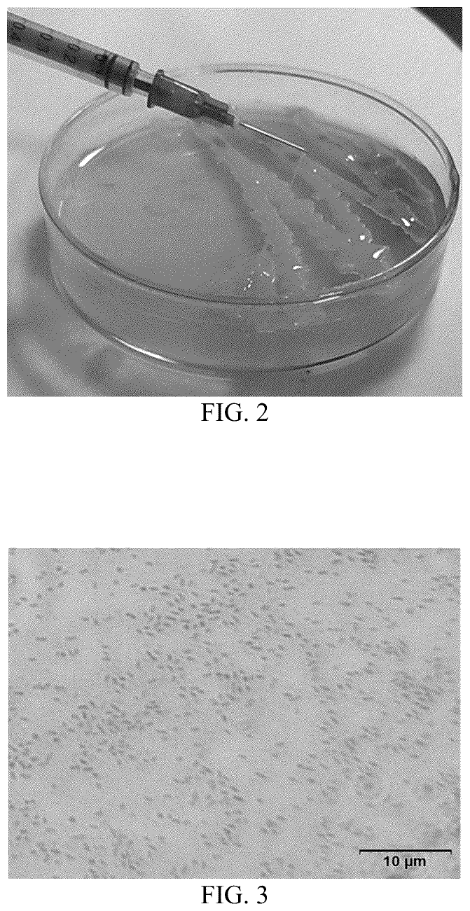

Research results showed that the strain thallus was in a short rod shape, had a single flagellum, can move, had raised bacterial colony and uneven edge, smooth and wet surface in a fusion state, as well as viscous wiredrawing polysaccharide on the bacterial colony surface. The cells were long rod-shaped, with the dimensions of (0.1−0.2)×(2-3) μm. The bacterial colony morphology was shown in FIG. 2, and the stained thallus morphology was shown in FIG. 3.

The physiological and biochemical characteristics of Pseudomonas Pse-1 were as follows: aerobic, growth temperature within a range of 37-45° C., the most suitable growth temperature of 37° C., growth pH range of 5-10, optimal growth pH range of 7.8-8, and the NaCl concentration tolerance range within a range of 0-10 wt. %.

### Example 2

The performance parameters such as surface tension of the fermentation broth, oil sand washing performance and apparent viscosity of the fermentation broth under the oil reservoir condition of the Pseudomonas Pse-1, improved recovery ratio by the physical simulation experiment were evaluated. Wherein the oil reservoir temperature was 60° C.

The performance evaluation method of Pseudomonas Pse-1 comprised the following steps:

(1) Measurements of Surface Tension and Apparent Viscosity of the Fermentation Broth with Different Dilution Ratios Under the Oil Reservoir Conditions

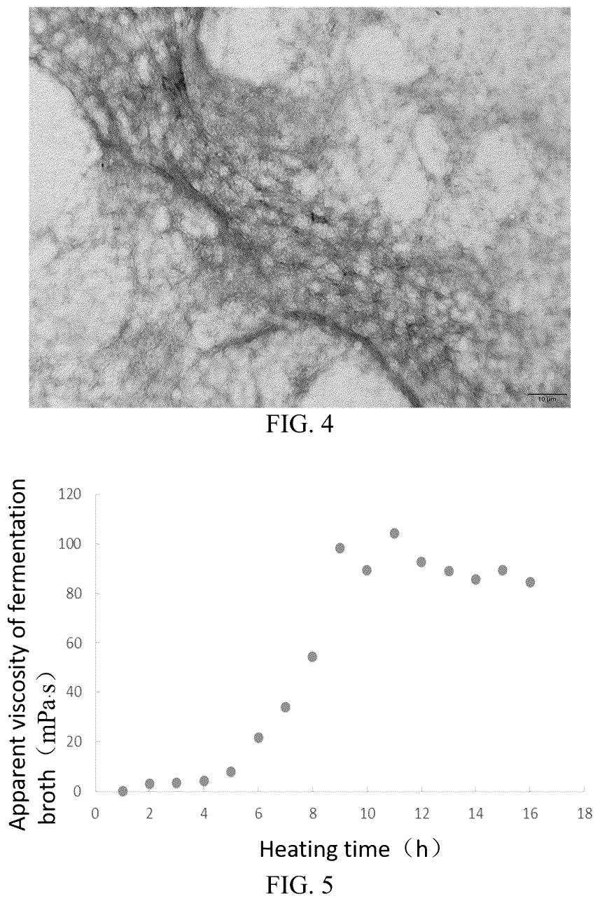

The Pse-1 fermentation broth was diluted to varying degrees with the formation water (mineralization degree of 18,712 mg/L) (dilution degree=volume of formation water/(volume of fermentation broth+volume of formation water)×100%), the diluted fermentation broths were heated at the reservoir temperature for 8 h, then the surface tension at 25° C. and apparent viscosity at 60° C. of the fermentation broths having different dilution degrees were detected (the surface tension test was performed with reference to the flat plate method test standard of the China National Standard GB/T22237-2008, the apparent viscosity was measured by using the Brook field rotational viscometer (rotor model: rotor No. 3, rotor speed: 60 r/min) (refer to the viscosity test method stipulated in the China National Standard GB 1886.41-2015), the microscopic observation results of the fermentation broth with 50% dilution degree were shown in FIG. 4, the viscosity change of the fermentation broth with 50% dilution degree after heated for different times was shown in FIG. 5, and the measurement results when heated for 8 h were illustrated in Table 1 below.

TABLE 1

| Dilution | Surface tension | Apparent viscosity | Oil washing |
|---|---|---|---|
| degree | (mN/m) | (mPa · s) | efficiency (%) |
| 10% | 24.7 | 150 | 96.4 |
| 20% | 25.2 | 121 | 95.2 |
| 30% | 25.4 | 107 | 93.1 |
| 40% | 26.7 | 86 | 92.4 |
| 50% | 28.3 | 65 | 91.5 |
| 60% | 35.7 | 24 | 84.2 |

### (2) Evaluation of Oil Sand Washing Performance of the Fermentation Broth

The oil sand preparation and standard curve plotting were carried out according to the Enterprise Standard Q/SH 1020 1518-2013 stipulated by the Shengli Oil Field. 3 g of oil sand was weighed and placed in a colorimetric tube with a volume of 50 mL, which was labeled as A.

The Pse-1 fermentation broth was heated at the oil reservoir temperature for 8 h, and then diluted according to the different proportions.

10 mL of the treated and diluted Pse-1 fermentation broth was added into a colorimetric tube, covered with a cap, the colorimetric tube was placed in a thermostatic water bath at the temperature of 60° C., and the colorimetric tube was taken out at an interval of 15 min, slightly shook for 10 times, then placed in the water bath, the colorimetric tube was taken out after 1 h of water bath.

The supernatant was carefully poured out, the residual bacteria solution in the colorimetric tube was repeatedly washed with distilled water until the eluate was transparent, and the supernatant was carefully poured out.

Another colorimetric tube with a volume of 50 mL was taken, which was labeled as B and was placed on a colorimetric tube holder, and a funnel was inserted into the colorimetric tube.

The colorimetric tube A was placed directly above the funnel with the mouth inclined downwards, the bottom of said colorimetric tube A was rinsed with a washing bottle filled with distilled water, and all the oil sand in said colorimetric tube A was carefully transferred to the colorimetric tube B, and the upper layer washing liquid was poured out.

The colorimetric tube with the oil sand was placed in an oven for drying at 105° C. for 4 h, and the colorimetric tube was taken out, then put in a dryer and cooled to room temperature. A suitable amount of petroleum ether with a boiling range of 60-90° C. was added into the colorimetric tube with the oil sand, fully shaken, and diluted to the scale. The petroleum ether solution was absorbed, the absorbance was measured on a spectrophotometer, and the residual oil content in the colorimetric tube was investigated according to the Standard Curve. The oil washing efficiency X=(1−W₁/KW₀)×100%, wherein W₁ denoted the residual oil content of the oil sand in the colorimetric tube, K denoted the mass fraction of the oil contained in the oil sand, and W₀ denoted the mass of the oil sand.

The oil washing efficiency X at the dilution degree of 40%=(1−0.0046/0.02×3.0067)×100%=92.4%, and the results of the final evaluation of the oil washing efficiency of the fermentation broths at different dilution degrees were shown in Table 1 above.

### (3) Evaluation of Improved Recovery Ratio by the Physical Simulation Experiment

\textcircled{1} Preparation of a core: a rock core was filled and sterilized, and the air permeability was measured;
\textcircled{2} the rock core was vacuumized, and saturated with the simulated formation water, and the PV (pore volume) of the rock core was measured;
\textcircled{3} the rock core was saturated with crude oil, then subjected to aging for 7 d, and the irreducible water saturation was calculated;
\textcircled{4} the primary water flooding was performed until the water content of the produced water was more than 98%, and the recovery ratio of the primary water flooding was calculated;
\textcircled{5} about 0.3 PV of Pseudomonas Pse-1 fermentation broth with a dilution degree of 50% was injected, both an injection end and a production end were closed, and denatured at the oil reservoir temperature for about 9 h;
\textcircled{6} the secondary water flooding was performed until the water content of the produced water was 100%, and the improved recovery ratio of Pseudomonas Pse-1 was calculated, which reached 23%. The above experiments indicated the properties of Pseudomonas Pse-1: the surface tension of a fermentation liquid under the dilution degree of less than or equal to 50% was lower than 30 mN/m, the viscosity after denaturing was more than 50 mPa·s, the oil washing efficiency was larger than 90%; the increased recovery ratio by physical simulation experiment was 21% (compared to the pure water flooding).

### Example 3

### Preparation of Pseudomonas Pse-1 Used as an Oil-Extraction Bacterial Solution.

A seed solution was firstly obtained according to the method above, the seed solution was then inoculated into a nutrient medium for culture to obtain a fermentation solution, wherein the inoculation age refers to the culture time during the preparation of the seed solution:

In Example 3.1, the nutrient medium comprised 10 g/L of xylose, 30 g/L of glycerol, 2 g/L of sodium nitrate, 2 g/L of dipotassium hydrogen phosphate, 2 g/L of sodium chloride, 0.1 g/L of calcium chloride, 0.5 g/L of magnesium sulfate, 1 g/L of yeast extract powder, and the pH was 7.8.

In Example 3.2, the nutrient medium comprised 12 g/L of xylose, 35 g/L of glycerol, 4 g/L of sodium nitrate, 3 g/L of dipotassium hydrogen phosphate, 3 g/L of sodium chloride, 0.1 g/L of calcium chloride, 0.7 g/L of magnesium sulfate, 2 g/L of yeast extract powder, the pH was 7.5, and the balance was water.

In Example 3.3, the nutrient medium comprised 8 g/L of xylose, 15 g/L of glycerol, 1 g/L of sodium nitrate, 1 g/L of dipotassium hydrogen phosphate, 1 g/L of sodium chloride, 0.05 g/L of calcium chloride, 0.3 g/L of magnesium sulfate, 0.5 g/L of yeast extract powder, the pH was 8, and the balance was water.

In Example 3.1, the fermentation conditions for the Pseudomonas were as follows: the inoculation amount was 2 vol %, the inoculation age was 12 h, the initial pH was 7.5, the temperature was 37° C., the stirring speed was 220 rpm, the ventilation amount was 1 L/min, and the fermentation time was 72 h.

In Example 3.2, the fermentation conditions for the Pseudomonas were as follows: the inoculation amount was 3 vol %, the inoculation age was 24 h, the initial pH was 7.5, the temperature was 40° C., the stirring speed was 200 rpm, the ventilation amount was 1.5 L/min, and the fermentation time was 60 h.

In Example 3.3, the fermentation conditions for the Pseudomonas were as follows: the inoculation amount was 1 vol %, the inoculation age was 12 h, the initial pH was 8, the temperature was 45° C., the stirring speed was 160 rpm, the ventilation amount was 2 L/min, and the fermentation time was 48 h.

The performance parameters of the above fermentation broths under the dilution degree condition of 50% were shown in Table 2 below.

TABLE 2

| | | | | Improved recovery |
|---|---|---|---|---|
| | | Apparent | Oil washing | ratio by physical |
| | Surface tension | viscosity | efficiency | simulation |
| Examples | (mN/m) | (mPa · s) | (%) | experiment (%) |
| Example | 27.6 | 68 | 92.3 | 24 |
| 3.1 | | | | |
| Example | 28.3 | 65 | 91.5 | 23 |
| 3.2 | | | | |
| Example | 28.4 | 67 | 91.8 | 22 |
| 3.3 | | | | |

### Example 4

The Example illustrated the use of the Pseudomonas Pse-1 fermentation broth provided by the invention in a thickened oil well A1 in a block of the Shengli Oil Field in China.

Overview of the oil well: the formation temperature was 65° C., the mineralization degree of formation water was 18,712 mg/L, the effective thickness of an oil layer was 6.7 m, the porosity was 35%, the permeability was 567×10⁻³ μm², and the viscosity of the dehydrated and degassed crude oil at 50° C. was 3,764 mPa·s.

The use of the Pseudomonas Pse-1 fermentation broth in the microbial huff and puff. The microbial huff and puff was to inject Pseudomonas Pse-1 fermentation broth into a stratum through an oil well, and the single-well productivity was improved by means of the synergistic effect of a biosurfactant and a temperature-sensitive viscoelastic biological polysaccharide.

The specific implementation steps were as follows: the prepared Pseudomonas Pse-1 fermentation broth with a dilution degree of 30% was injected into a stratum through tubing-casing annulus at an injection speed of 15 m³/h.

The injection volume of the fermentation broth was calculated according to the effective thickness, porosity and treatment radius of the oil layer of the oil well.

Formula of the total injection volume: V=3.14R²Hϕα, wherein
V denoted the total injection volume, the unit was m³;
R denoted the treatment radius, 7 m;
H denoted the effective thickness (6.7 m);
ϕ denoted the porosity (the value was 0.35);
β denoted the dosage coefficient (the value was 1.1).

The used amount of the Pseudomonas Pse-1 fermentation broth with a dilution degree of 30% was calculated as 397 m³ according to the formula; the pump truck was used for injecting the prepared Pse-1 fermentation broth into the stratum through the tubing-casing annulus; the oil well was closed and cultured for 30 d, the well was then opened for production.

Test results: a water-flooding mode was adopted for oil extraction before the implementation. After the oil well A1 was opened for production, the date when the daily oil yield of the oil well was larger than that before the implementation may be denoted as the start date of the validity period, both the fluid production and the oil production increased than those before the implementation, the average daily oil yield of a single well increased by 2.8 t, the cumulative increased oil production was 980 t, the lowest viscosity of the crude oil was reduced to 762 mPa·s, the decreasing amplitude reached 80%, the date when the daily oil yield of the oil well was less than that before the implementation may be denoted as the end date of the validity period, the validity period reached 350 d, the output-input ratio was 5.7, and the field test effects of the Pseudomonas Pse-1 were desirable.

### Example 5

The Example illustrated the use of the Pseudomonas Pse-1 fermentation broth provided by the invention in a thickened oil well B1 in a block of the Shengli Oil Field in China.

Overview of the oil well: the formation temperature was 60° C., the mineralization degree of formation water was 22,140 mg/L, the effective thickness of an oil layer was 5.1 m, the porosity was 30%, the permeability was 467×10⁻³ μm², and the viscosity of the dehydrated and degassed crude oil at 50° C. was 1,875 mPa·s.

The use of the Pseudomonas Pse-1 fermentation broth in the microbial huff and puff. The microbial huff and puff was to inject Pseudomonas Pse-1 fermentation broth into a stratum through an oil well, and the single-well productivity was improved by means of the synergistic effect of a biosurfactant and a temperature-sensitive viscoelastic biological polysaccharide.

The specific implementation steps were as follows: the prepared Pseudomonas Pse-1 fermentation broth with a dilution degree of 50% was injected into a stratum through the tubing-casing annulus at an injection speed of 10 m³/h.

The injection volume of the fermentation broth was calculated according to the effective thickness, porosity, and treatment radius of the oil layer of the oil well.

The formula of the total injection volume: V=3.14R²Hϕβ, wherein
V denoted the total injection volume, the unit was m³;
R denoted the treatment radius, 6 m;
H denoted the effective thickness (5.1 m);
denoted the porosity (the value was 0.3);
β denoted the dosage coefficient (the value was 1.2).

The used amount of the Pseudomonas Pse-1 fermentation broth with a dilution degree of 50% was calculated as 208 m³ according to the formula; the pump truck was used for injecting the prepared Pse-1 fermentation broth into the stratum through the tubing-casing annulus; the oil well was closed and cultured for 20 d, the well was then opened for production.

Test results: a water-flooding mode was adopted for oil extraction before the implementation. After the oil well B1 was opened for production, the date when the daily oil yield of the oil well was larger than that before the implementation may be denoted as the start date of the validity period, both the fluid production and the oil production increased than those before the implementation, the average daily oil yield of a single well increased by 3.7 t, the cumulative increased oil production was 1,166 t, the lowest viscosity of the crude oil was reduced to 283 mPa·s, the decreasing amplitude reached 84.9%, the date when the daily oil yield of the oil well was less than that before the implementation may be denoted as the end date of the validity period, the validity period reached 315 d, the output-input ratio was 6, and the field test effects of the Pseudomonas Pse-1 were desirable.

### Example 6

The Example illustrated the use of the Pseudomonas Pse-1 fermentation broth provided by the invention in an oil well Cl in a block of the Shengli Oil Field in China, the oil group comprised 1 well for injection and 5 wells for extraction.

Overview of the oil well: the formation temperature was 65° C., the mineralization degree of formation water was 24,134 mg/L, the effective thickness of an oil layer was 4.7 m, the porosity was 0.32, the permeability was 653×10⁻³ μm², the total pore volume of the oil well group was 8.75×10+m³, the viscosity of the dehydrated degassed crude oil at 50° C. was 2,719 mPa·s, and the well spacing was 230 m.

The use of the Pseudomonas Pse-1 fermentation broth in the microbial oil displacement. The microbial oil displacement was implemented by injecting the Pseudomonas fermentation broth through a water injection well in a slug mode, and the productivity of an oil well in the oil well group was improved by utilizing the metabolite of the Pseudomonas fermentation broth Pse-1.

The microbial oil flooding adopted a slug alternate injection mode, the total injection volume of the oil-extraction bacterial solution was 0.05 PV in a total volume of 4,350 m³, and the total water injection volume was 0.1 PV in a total volume of 8,700 m³.

The method comprised the following specific steps:
(1) The oil-extraction bacterial solution and water were injected into an oil reservoir in sequence, wherein the injection volume of the oil-extraction bacterial solution was 621 m³, the injection speed was 20 m³/d, the injection volume of water was 828 m³, and the injection speed was 30 m³/d;
(2) stopping injection operation for 1 d;
(3) the injected water was 414 m³, so that the water injection of one period was completed.

Test results: a water-flooding mode was adopted for oil extraction before the implementation. The well group was injected for a total of 7 rounds, the date when the peak daily oil yield of the oil well after a single round of injection was larger than that before the implementation may be denoted as the start date of the validity period, both the fluid production and the oil production for each oil well after the injection increased than those before the implementation, the average daily oil yield of a single well increased by 6.3 t, the cumulative increased oil production was 13,261 t, the lowest viscosity of the crude oil was reduced to 316 mPa·s, the decreasing amplitude reached 88%, the date when the peak daily oil yield of the oil well after a single round of injection was less than that before the implementation may be denoted as the end date of the validity period, the validity period reached 421 d, the output-input ratio was 4.2, and the field test effects were desirable.

### Example 7

The Example illustrated the use of the Pseudomonas Pse-1 fermentation broth provided by the invention in the oil well DI in a block of the Shengli Oil Field in China, the oil group comprised 1 well for injection and 4 wells for extraction.

Overview of the oil well: the formation temperature was 72° C., the mineralization degree of formation water was 19,473 mg/L, the effective thickness of an oil layer was 5.7 m, the porosity was 35%, the permeability was 429× 10⁻³ μm², the total pore volume of the oil well group was 3.4×10⁻³ m³, the viscosity of the dehydrated degassed crude oil at 50° C. was 3,271 mPa·s, and the well spacing was 270 m.

The microbial oil flooding adopted a slug alternate injection mode, the total injection volume of the oil-extraction bacterial solution was 0.1 PV in a total volume of 3,400 m³, and the total water injection volume was 0.15 PV in a total volume of 5,100 m³.

The method comprised the following specific steps:
(1) The oil-extraction bacterial solution and water were injected into an oil reservoir in sequence, wherein the injection volume of the oil-extraction bacterial solution was 595 m³, the injection speed was 20 m³/d, the injection volume of water was 531 m³, and the injection speed was 40 m³/d;
(2) stopping injection operation for 2 d;
(3) the injected water was 318 m³, and the water injection speed was 50 m³/d such that the water injection of one period was completed.

Test results: a water-flooding mode was adopted for oil extraction before the implementation. The well group was injected for a total of 6 rounds, the date when the peak daily oil yield of the oil well after a single round of injection was larger than that before the implementation may be denoted as the start date of the validity period, both the fluid production and the oil production for each oil well after the injection increased than those before the implementation, the average daily oil yield of a single well increased by 5.5 t, the cumulative increased oil production was 8,316 t, the lowest viscosity of the crude oil was reduced to 215 mPa·s, the decreasing amplitude reached 93%, the date when the peak daily oil yield of the oil well after a single round of injection was less than that before the implementation may be denoted as the end date of the validity period, the validity period reached 378 d, the output-input ratio was 4.7, and the field test effects were desirable.

The above content describes in detail the preferred embodiments of the invention, but the invention is not limited thereto. A variety of simple modifications can be made in regard to the technical solutions of the invention within the scope of the technical concept of the invention, including a combination of individual technical features in any other suitable manner, such simple modifications and combinations thereof shall also be regarded as the content disclosed by the invention, each of them falls into the protection scope of the invention.

1. A Pseudomonas (Pseudomonas sp.) strain, characterized in that its preservation number is CGMCC No. 22583.

2. (canceled)

3. A method for recovering oil, characterized in that the method comprises the following steps: injecting an oil-extraction bacterial solution into an oil reservoir to produce oil, wherein the oil-extraction bacterial solution contains at least one selected from the group consisting of a thallus of the Pseudomonas according to claim 1, an extracellular metabolite of the Pseudomonas, and an intracellular metabolite of the Pseudomonas.

4. The method according to claim 3, wherein the oil-extraction bacterial solution has a surface tension at 25° C. being less than or equal to 30 mN/m; and an apparent viscosity at 60° C. being larger than or equal to 50 mPa·s.

5. The method according to claim 3, wherein the method further comprises the steps of preparing an oil-extraction bacterial solution: (i) inoculating the Pseudomonas into a nutrient medium for fermentation; and optionally, (ii) subjecting the resulting fermentation product to a thallus lysis operation, or separating the thallus from the fermentation product and subjecting the obtained thallus to lysis.

6. The method according to claim 5, wherein the nutrient medium comprises a carbon source, a nitrogen source, and an inorganic salt, and the initial pH of the nutrient medium is within a range of 7.5-8.

7. The method according to claim 6, wherein the nutrient medium contains the carbon source in an amount of 20-50 g/L;
and/or, the nutrient medium contains the nitrogen source in an amount of 0.5-2 g/L;
and/or, the nutrient medium contains the inorganic salt in an amount of 3-12 g/L.

8. The method according to claim 6, wherein the carbon source is xylose and/or glycerol;
and/or, the nitrogen source is yeast extract powder;
and/or, the inorganic salt comprises at least one selected from the group consisting of sodium salt, potassium salt, calcium salt, and magnesium salt.

9. The method according to claim 5, wherein the nutrient medium comprises 8-12 g/L of xylose, 15-35 g/L of glycerol, 1-4 g/L of sodium nitrate, 1-3 g/L of dipotassium phosphate, 1-3 g/L of sodium chloride, 0.05-0.1 g/L of calcium chloride, 0.3-0.7 g/L of magnesium sulfate, 0.5-2 g/L of yeast extract powder, and an initial pH of the nutrient medium is within a range of 7.5-8.

10. The method according to claim 5, wherein the fermentation conditions comprise: the inoculation amount is within a range of 10⁶-10⁸ CFU/mL, the fermentation temperature is within a range of 37-45° C., and the fermentation time is within a range of 48-72 h.

11. The method according to claim 3, wherein the mode of injecting an oil-extraction bacterial solution into an oil reservoir to produce oil is an oil well huff and puff, and the oil well huff and puff has a closed well cultivation time of 15-30 d.

12. The method according to claim 11, wherein the total injection volume of the oil-extraction bacterial solution satisfies the formula V=πR²Hϕβ, wherein:
V denotes the total injection volume of the oil-extraction bacterial solution, the unit is m³;
R denotes the treatment radius, the unit is m;
H denotes the effective thickness, the unit is m;
ϕ denotes the porosity;
β denotes the dosage coefficient, the value is within a range of 1-1.5.

13. The method according to claim 3, wherein the mode of injecting an oil-extraction bacterial solution into an oil reservoir to produce oil is the microbial flooding, comprising:
(1) injecting an oil-extraction bacterial solution and water into an oil reservoir in sequence, wherein the used amount of the oil-extraction bacterial solution in the step is 14-50% of the total injection volume of the oil-extraction bacterial solution;
(2) stopping the injecting operation;
(3) injecting water into the oil reservoir, wherein the ratio of the water injection volume in step (1) to the water injection volume in step (3) is 1:0.05-0.7;
(4) circularly performing the steps (1)-(3) at least twice.

14. The method according to claim 13, wherein the total injection volume of the oil-extraction bacterial solution is within a range of 0.05-0.1 PV, and the total injection volume of water is within a range of 0.1-0.3 PV, wherein PV denotes the pore volume of the reservoir stratum where the oil reservoir is located;
and/or, the circular times performed in step (4) is 2-7.

15. The method according to claim 3, wherein the viscosity of the dehydrated and degassed crude oil in the oil reservoir at 50° C. is less than or equal to 10,000 mPa·s.

16. The method according to claim 4, wherein the oil-extraction bacterial solution has a surface tension at 25° C. within a range of 24.5-28.5 mN/m; and an apparent viscosity at 60° C. within a range of 65-150 mPa·s.

17. An oil-extraction bacterial solution, wherein the oil-extraction bacterial solution comprises the Pseudomonas of claim 1 and/or metabolite thereof.


SEQUENCE LISTING (1 sequence)

SEQ ID NO: 1 - DNA, 1423 bp, Pseudomonas sp.
  source 1..1423: mol_type = genomic DNA
agcctacacatgcaagtcgagcggcagcacgggtacttgtacctggtggcgagcggcggacgggtgagtaatgcctaggaatctgcctggtagtgggggataacgctcggaaacggacgctaataccgcatacgtcctacgggagaaagcaggggaccttcgggccttgcgctatcagatgagcctaggtcggattagctagttggtgaggtaatggctcaccaaggcgacgatccgtaattggtctgagaggatgatcagtcacactgcaactgagacacggtccagactcctacgggaggcacgagtggggaatattggacaatgggcgaaagcctgatccagccatgccgcgtgtgtgaagaaggtgtacggattgttaagcacaataagttgggaggaagggcatttacctaatacgtaagtgttttgacgttaccgacagaaaaaccaccaactacctctgtgccagcagccgcggtaatacagagggtgcaagcgttaatcgcaataactgggcgtaaagcgcgcgtaggtggtatgttaagttggatgtgaaatccccgggctcaacctgggaactgcatccaaaactcccaagctagactatggtagagggtggtggaatttcctgtgtagcggtgatatccgtagatatcggaaggaacaccagtggtgaaggcgaccacctggactgatactgacactgaggtgcgaaagcgtgcggagcatacaggattagaaaccctggtagtccacgccgtaaacgatgtcaactagccgttgggtgccttgagctcttcgtggcgcaggtatcgcattaagttgaccgcctggggagtacggccgcaaggttataactcatatgatttgacgggggggcgcacaagcggtggagcatgtggtttaattcgaagcaacgcgaagaaccttaccaggccttgacatccaataaactttccagagatggattggtgccttcgggaacattgagacaggtggtgcatgcctgtcgacagctcgtgtcgtgagatgttgggttaagtcccgtaacgagcgcaacccttgtccttagttaccagcacattttggtccccactctaaggagactgccggtgacaaaccggaggaaggtggggatgacctcaagtcatcatggcccttacggcctgggctacacacgtgctacaatggtcggtacagagggttgccaagccgcgaggtagagctaatcccataaaaccgatcgtagtccggatcgcagtctgcaactcgactgcgtgaagtcggaatcgctagtaatcgcgtttcagaatgtcgcggtgaatacgttcccgggccttgtacacaccgcccgtcacaccatccgagtgggttgcaccagaagtagctagtctaaccttcggggggacggtaccacggt